(19) United States
(12) Patent Application Publication
(10) Pub. No.: US 2025/0045914 A1
(43) Pub. Date: Feb. 6, 2025

(54) SYSTEM AND METHOD FOR PROCESSING BRAIN SCAN INFORMATION TO AUTOMATICALLY IDENTIFY ABNORMALITIES

(71) Applicant: KONINKLIJKE PHILIPS N.V., EINDHOVEN (NL)

(72) Inventors: FABIAN WENZEL, HAMBURG (DE); NICK FLÄSCHNER, HAMBURG (DE); ARNE EWALD, HAMBURG (DE); ELIZA TEODORA ORASANU, HAMBURG (DE)

(21) Appl. No.: 18/717,630

(22) PCT Filed: Dec. 2, 2022
(86) PCT No.: PCT/EP2022/084228

§ 371 Date: Jun. 7, 2024

Related U.S. Application Data
(60) Provisional application No. 63/287,998, filed on Dec. 10, 2021.

(51) Int. Cl. G06T 7/00; G06T 5/40; G06V 10/44; G06V 10/764; G16H 30/20

(52) U.S. Cl. CPC G06T 7/0012; G06T 5/40; G06V 10/44; G06V 10/764; G16H 30/20; G06T 2207/30016; G06T 2207/30096

(57) ABSTRACT

A method for processing medical information includes receiving an image slice of a brain including segmented regions, forming a first histogram of intensity values for the image slice, and forming a second histogram of intensity values for the image slice. A difference histogram is then generated based on the first and second histograms and the existence of an abnormality in the image slice is determined based on the difference histogram. The first histogram may correspond to a first segmented region in a first portion of the image slice, and the second histogram may correspond to a second segmented region in a second portion of the image slice which is complementary to the first portion. The first and second segmented regions may be, for example, segmented and labeled ASPECTS regions.

One or more embodiments described herein relate to processing information to automatically identify abnormalities in brain scans.

### BACKGROUND

Healthcare professionals are continually challenged to find new ways of providing critical care to patients who suffer from brain-related conditions. The ability to detect these conditions quickly and accurately may guide the course of treatment with the hope of saving greater numbers of lives, especially for those who have suffered severe conditions such as ischemic stroke.

When a person is suspected of having certain types of a brain abnormalities, the clinical course of action is usually to obtain a computed tomography (CT) image of the brain. The CT image is then assessed by a radiologist or other professional, which in some cases may involve generating an Alberta Stroke Program Early CT Score (ASPECTS). This score indicates the severity of the condition the patient has suffered. ASPECTS scores range from 0 to 10, with scores of 6 or greater qualifying for procedures such as mechanical thrombectomy.

As indicated, ASPECTS scores are generated based on visual assessments. These assessments are often inaccurate for a variety of reasons, e.g., misreading of the images by the radiologist, head tilt or movement of the patient during scanning and uncertainties respecting the extent of anatomical regions and subtle contrast changes. Manual assessments of brain scan images are subject to significant delays. These and other reasons prevent patients from receiving competent care, which, in turn, may lead to complications which otherwise could have been prevented had an expert system been used to provide a more effective analysis of brain scan assessments automatically and in real time, at least from the point of acquisition of the images.

### SUMMARY

Embodiments described herein provide a system and method which automatically perform brain scan assessments to locate abnormalities in patients who are suspected of having abnormalities, including but not limited to ischemic lesions caused by stroke.

In accordance with one or more embodiments, a method for processing medical information includes receiving an image slice of a brain including segmented regions; forming a first histogram of intensity values for the image slice; forming a second histogram of intensity values for the image slice; and determining an abnormality in the image slice based on the first histogram and the second histogram, wherein the first histogram corresponds to a first segmented region in a first portion of the image slice and the second histogram corresponds to a second segmented region in a second portion of the image slice which is complementary to the first portion. The first segmented region and the second segmented region may be complementary ASPECTS regions.

The method may include generating at least one difference histogram based on the first histogram and the second histogram, wherein determining the abnormality in the image slice is based on the at least one difference histogram. Determining the abnormality may include identifying that the at least one difference histogram has values in a range; and determining the abnormality based on the values of the at least one difference histogram in the range.

Determining the abnormality may include identifying that the at least one difference histogram has one or more values that exceed a predetermined reference value; and determining the abnormality based on the one or more values of the at least one difference histogram exceeding the predetermined reference value. Generating the at least one difference histogram may include subtracting the intensity values of the first histogram from the intensity values of the second histogram.

Generating the at least one difference histogram may include generating a first difference histogram based on a difference between the first histogram and a first reference histogram; and generating a second difference histogram based on a difference between the second histogram and a second reference histogram. The first reference histogram may be indicative of brain tissue without a lesion in the first segmented region, and the second reference histogram may be indicative of brain tissue without a lesion in the second segmented region. Determining the abnormality may include generating a feature vector based on the at least one difference histogram; inputting the feature vector into a classifier model; and predicting the abnormality based on an output of the classifier model.

The method may include determining whether the first histogram has a concentration of intensity values in a predetermined range; and identifying that the image slice has an old abnormality when the first histogram has the concentration of intensity values in the predetermined range. The method may include generating the difference histogram based on an exclusion of the intensity values in the predetermined range of the first histogram corresponding to the old abnormality.

In accordance with one or more embodiments, a system for processing medical information, includes a histogram generator configured to generate a first histogram of intensity values for an image slice and a second histogram of intensity values for the image slice, the image slice including segmented regions of a brain; and a decision engine configured to determine an abnormality in the image slice based on the first histogram and the second histogram, wherein the first histogram corresponds to a first segmented region in a first portion of the image slice and the second histogram corresponds to a second segmented region in a second portion of the image slice which is complementary to the first portion.

The system may include difference logic configured to generate at least one difference histogram based on the first histogram and the second histogram, wherein the decision engine is configured to determine an abnormality in the image slice based on the at least one difference histogram.

The decision engine may be configured to determine the abnormality by: identifying that the at least one difference histogram has values in a range; and determining the abnormality based on the values of the at least one difference histogram in the range. The decision engine may be configured to determine the abnormality by: identifying that the at least one difference histogram has one or more values that exceed a predetermined reference value; and determining the abnormality based on the one or more values of the at least one difference histogram exceeding the predetermined reference value.

The difference logic may be configured to generate the at least one difference histogram by subtracting the intensity values of the first histogram from the intensity values of the second histogram. The difference logic may be configured to: generate a first difference histogram based on a difference between the first histogram and a first reference histogram; and generate a second difference histogram based on a difference between the second histogram and a second reference histogram. The first reference histogram may be indicative of brain tissue without a lesion in the first segmented region, and the second reference histogram may be indicative of brain tissue without a lesion in the second segmented region.

The decision engine may be configured to determine the abnormality by: generating a feature vector based on the at least one difference histogram; inputting the feature vector into a classifier model; and predicting the abnormality based on an output of the classifier model. The difference logic may be configured to generate the at least one difference histogram by subtracting the intensity values of the first histogram from the intensity values of the second histogram.

The system may include a discrimination logic configured to: determine whether the first histogram has a concentration of intensity values in a predetermined range; and identify that the image slice has an old abnormality when the first histogram has the concentration of intensity values in the predetermined range. The difference logic is configured to generate the at least one difference histogram based on an exclusion of the intensity values in the predetermined range of the first histogram corresponding to the old abnormality.

### BRIEF DESCRIPTION OF THE DRAWINGS

Additional objects and features of the invention will be more readily apparent from the following detailed description and appended claims when taken in conjunction with the drawings. Although several example embodiments are illustrated and described, like reference numerals identify like parts in each of the figures, in which:

FIG. 1 shows an embodiment of a medical image analyzer.

FIGS. 2A and 2B shows an embodiment of a method for analyzing medical images.

FIGS. 3A and 3B shows an example of segmented and labeled image slices.

FIG. 4 shows an example of a lesion in one region of an image slice.

FIG. 5 shows an embodiment for generating one type of difference histogram.

FIG. 6A shows an embodiment for generating another type of difference histogram, and FIG. 6B shows another depiction of the difference histogram in FIG. 6B.

FIG. 7 shows examples of difference histograms generated for multiple regions.

FIG. 8 shows an embodiment for identifying old and new lesions.

FIG. 9 shows examples of histogram curves for old and new lesions.

FIG. 10 shows an embodiment of a classifier model for analyzing brain scan images.

FIGS. 11A and 11B show an embodiment of a method of classifying a brain scan image.

FIG. 12 shows examples of the variability in image parameters that may be used during training of the classifier model.

FIG. 13 shows examples of the variability in image parameters that may be used during training of the classifier model.

FIG. 14 shows examples of the variability in image parameters that may be used during training of the classifier model.

FIG. 15 shows an embodiment of a medical image analyzer.

### DETAILED DESCRIPTION

It should be understood that the figures are merely schematic and are not drawn to scale. It should also be understood that the same reference numerals are used throughout the figures to indicate the same or similar parts.

The descriptions and drawings illustrate the principles of various example embodiments. It will thus be appreciated that those skilled in the art will be able to devise various arrangements that, although not explicitly described or shown herein, embody the principles of the invention and are included within its scope. Furthermore, all examples recited herein are principally intended expressly to be for pedagogical purposes to aid the reader in understanding the principles of the invention and the concepts contributed by the inventor(s) to furthering the art and are to be construed as being without limitation to such specifically recited examples and conditions. Additionally, the term, “or,” as used herein, refers to a non-exclusive or (i.e., and/or), unless otherwise indicated (e.g., “or else” or “or in the alternative”). Also, the various example embodiments described herein are not necessarily mutually exclusive, as some example embodiments can be combined with one or more other example embodiments to form new example embodiments. Descriptors such as “first,” “second,” “third,” etc., are not meant to limit the order of elements discussed, are used to distinguish one element from the next, and are generally interchangeable. Values such as maximum or minimum may be predetermined and set to different values based on the application.

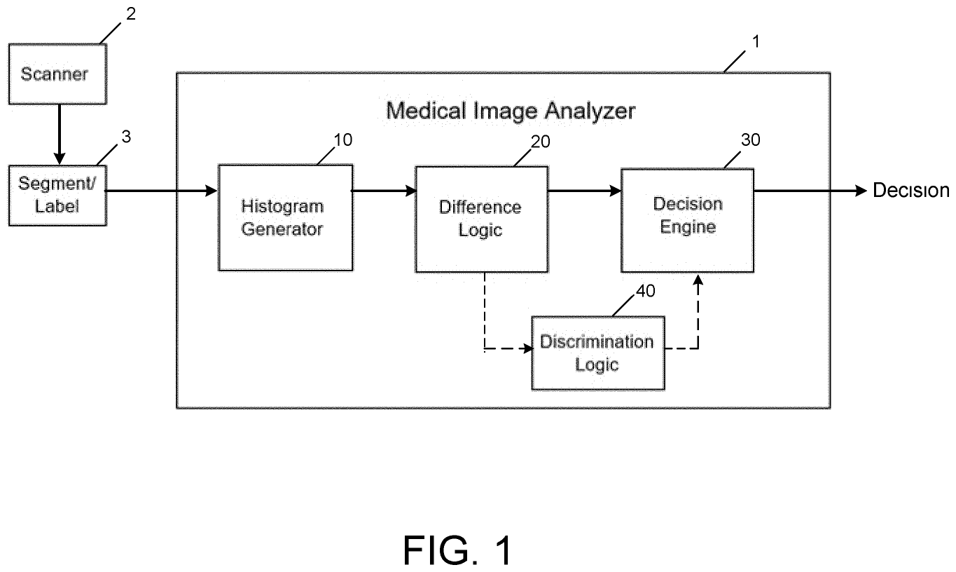

FIG. 1 illustrates an embodiment of a medical image analyzer 1 for automatically identifying abnormalities in the brain scans of patients. The system may identify the abnormalities in cooperation with image slices received from an imaging system such as a CT scanner 2. For example, for each patient the system may receive at least one image slice targeting a specific location of the brain wherein an abnormality is suspected. When ASPECTS regions are subject to analysis by the system, two image slices may be received, which image slices together account for all ten APSECTS regions on a per-hemisphere basis.

Referring to FIG. 1, the medical image analyzer includes a histogram generator 10, difference logic 20, and a decision engine 30. The histogram generator 10 may receive the image slice(s) from the CT scanner 2 as output from segmentation and labeling logic 3. The segmentation and labeling logic may analyze the image slice(s) to identify locations of ASPECTS regions and then superimpose contours and labels corresponding to those regions for input into the histogram generator. A more comprehensive discussion of how segmentation and labeling of the ASPECTS regions is performed in accordance with one or more embodiments in provided below.

For each image slice received, the histogram generator 10 may generate a first histogram of intensity values for a first segmented region and a second histogram of intensity values for a second segmented region of the image slice. The first and second segmented regions may be, for example, complementary ASPECTS regions on different lateral portions (e.g., left and right hemispheres) of the brain. In one embodiment, the intensity values may correspond to the full range of Hounsfield Units (HUs) or, in some embodiments, may correspond to a limited range of HU units as will be described. The histogram generator 10 may generate first and second histograms for just one complementary pair of ASPECTS regions, or in another embodiment may generate first and second histograms for a plurality of complementary pairs of ASPECTS regions including up to all ten regions.

The difference logic 20 is configured to generate a difference histogram based on the first histogram and the second histogram for one complementary pair of regions or for each complementary pair of regions when multiple pairs of regions are of interest. This may be accomplished, for example, by subtracting the HU values in the first histogram from the HU values from the second histogram. The first and second histograms will look significantly different from one another when one of the regions in the pair includes an abnormality. This is because the tissue affected by an abnormality will generate intensity values significantly different from healthy brain tissue. That difference will be reflected in the difference histogram.

The decision engine 30 is configured to determine an abnormality in one or more regions based on the difference histogram. This may be performed in a variety of ways. In one embodiment, the decision engine 30 may compare the values in the difference histogram to one or more predetermined reference values, ranges or patterns and then generate a decision as to whether an abnormality exists in at least one of the corresponding regions based on that comparison. In another embodiment, the decision engine 30 may implement a classifier model to predict the existence and location of an abnormality, at least within a certain probability. Embodiments of the operations performed by the decision engine will be explained in greater detail with reference to the method embodiments. Once a decision is made as to whether the image slice(s) includes an abnormality (which, for example may be within seconds of receiving the segmented and labeled image slices), healthcare professionals will be able to determine a course of treatment faster and more accurately that was able to be previously performed using manual techniques.

The medical image analyzer 1 may be modified in various ways to generate additional embodiments. For example, in one embodiment the difference logic 20 may compare the first histogram to a first reference histogram and the second histogram to a second reference histogram. The first reference histogram may have intensity values indicative of all normal brain tissue (e.g., health tissue or at least tissue without a lesion or a particular type of lesion) in the corresponding brain region. The second reference histogram may have intensity values indicative of normal healthy brain tissue (e.g., healthy tissue or at least tissue without a lesion or a particular type of lesion) in the corresponding complementary region, e.g., the same region on the opposing hemisphere. The difference logic 20 may then generate a first difference histogram based on a comparison of the first histogram and the first reference histogram (e.g., subtracting intensity values in the first histogram from intensity values in the first reference histogram), and a second difference histogram based on a comparison of the second histogram and the second reference histogram (e.g., subtracting intensity values in the second histogram from intensity values in the second reference histogram).

The decision engine 30 may then generate two decisions, a first decision indicating whether the region corresponding to the first difference histogram has an abnormality and a second decision separately indicating whether the region corresponding to the second difference histogram has an abnormality. This embodiment of the system may be able to pinpoint more quickly or with greater accuracy, for example, stroke lesions that are old or ones that are new in respective the regions subject to evaluation based on respective ones of the first and second histograms.

In another embodiment, the system may extend the regions of interest from 2D (ASPECT areas) to 3D (volumes containing voxels in different (e.g., adjacent) image slices that also belong to the ASPECT region. This embodiment ensures that the histograms caused by lesions that are more apparent in the vicinity (below or above) the 2D ASPECT area. The system and method embodiments may then be applied in an analogous manner to these 3D image slices.

In one embodiment, the medical image analyzer may include discrimination logic 40 for identifying and then excluding old lesions the difference histogram data to be input into the decision engine 30. Identifying and then excluding old lesions will allow the difference histogram(s) to include data only corresponding to the new lesions, which will allow radiologists and physicians to target treatment in a more effective manner. These and other operations relating to discriminating between new and old lesions as performed by the discrimination logic are discussed in greater detail below.

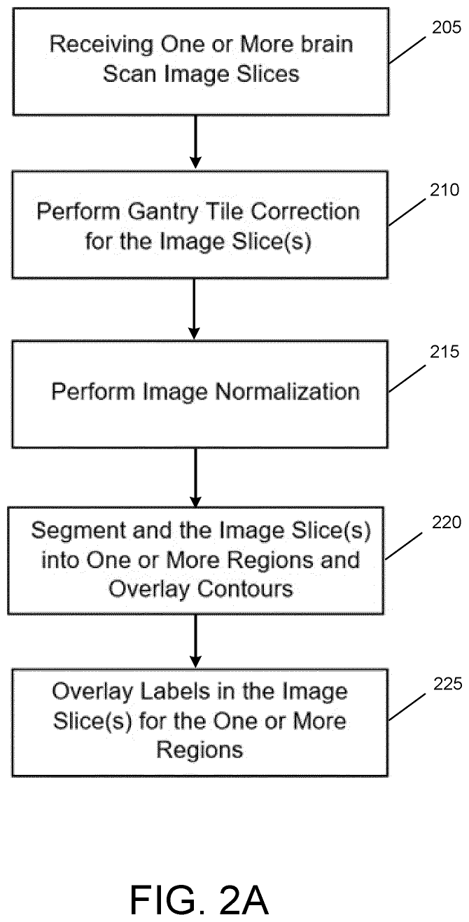

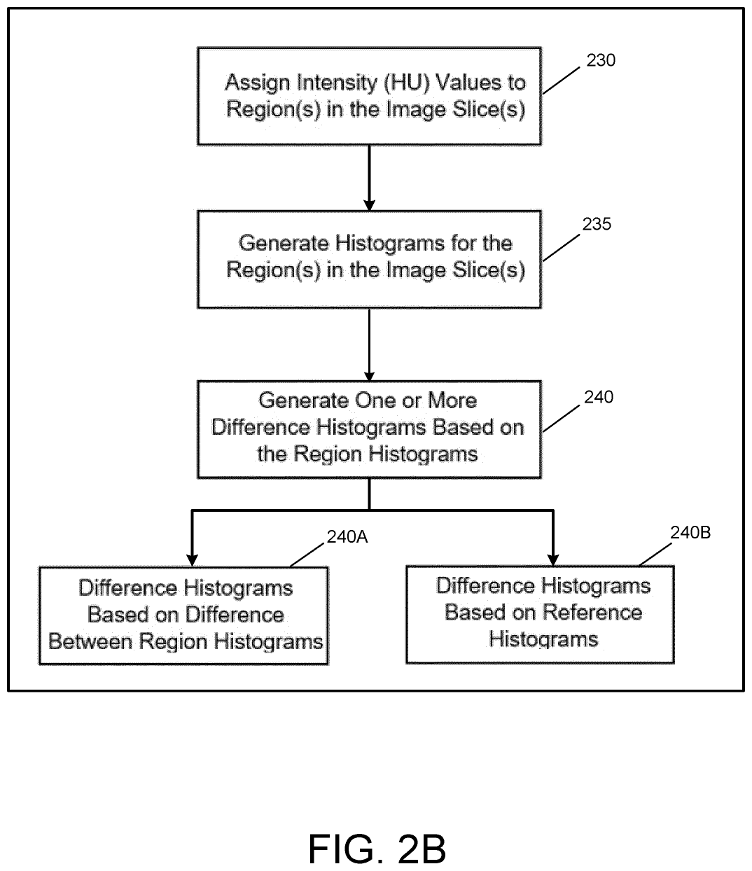

FIGS. 2A and 2B show operations included in an embodiment of a method for analyzing medical images to automatically identify abnormalities in the brain scans. The method may be performed by any of the system embodiments described herein or may be performed by a different system. For illustrative purposes, the method will be described as being performed by the system of FIG. 1.

Referring to FIG. 2A, the method includes, at 205, receiving one or more brain scan images of a patent to be evaluated, e.g., one suspected of having a brain abnormality such as a ischemic stroke or other brain condition. The images are generated by a scanning system, which, for example, may be a computed tomography scanner. In this case, the brain scan images may be non-contrast CT (NCCT) images. The CT scanner may be local (e.g., in the same hospital or clinical setting) or remotely located from the processing logic of the system implementing the method. In this latter case, the system and method embodiments may be used in an outsourcing context where, for example, the images are received from a network.

The brain scan images may correspond to a particular area of the brain. Given an axis passing longitudinally through the body of the patient (e.g., in a direction from head to toe), the one or more images may be of one or more axial slices that include target locations of the brain. The target locations (or slices) may be ones where one or more types of brain abnormalities are expected to occur given, for example, a suspected condition of the patient. In one embodiment, two images may be received that correspond to axial slices at different parts of the brain, e.g., a superior slice and an inferior slice.

When the patient is suspected of having suffered an ischemic stroke, the slices in the brain scan images may include, for example, a region including the middle cerebral artery (MCA). As will be described in greater detail, such a stroke will produce an occlusion (e.g., an ischemic lesion) in the image at the location of the MCA, at least with respect to the hemisphere of the brain where the stroke occurred. For purposes of illustration, the method will be described based on receiving the two image slices of the brain corresponding to the inferior slice and superior slice, but different image slices may be received and evaluated in accordance with the embodiments described herein. The image slices may be stored in a memory of the system for processing.

At 210, gantry tilt correction may be performed in the images. Gantry tilt relates to an aspect of a helical-scanning CT system equipped with a multi-row detector operating at some gantry tilting angle. The tilt angle may cause distortions in the image slices that can impair artifacts and other features having clinical significance from being clearly or accurately viewed. The image slices may therefore be pre-processed to reduce or filter out any distortions caused by gantry tilt angle. This may be accomplished, for example, by reformatting each of the image slices to produce a rectangular volume without gantry tilt.

At 215, the image slices may undergo intensity normalization. This may, for example, improve the process of assigning intensity values to the pixels in the image slices in a subsequent operation.

In one embodiment, normalizing the intensity of the brain scan images may be performed by adding a predetermined offset value (e.g., an offset value of 4,000) to the HU values in the image slices under evaluation. This type of normalization may cause the HU values to be concentrated throughout a much smaller range, which may serve to accentuate differences between regions in the slices that may allow for more accurate identification of abnormalities.

At 220, a segmentation operation is performed to identify different regions of interest in the image slices. This operation may involve segmenting each of the image slices into predetermined regions, such as, for example, the regions defined by the ASPECTS protocol. When segmentation is performed according to the ASPECTS protocol, a total of twenty regions of interest are defined: ten regions on the left hemisphere of the brain and ten regions on the right hemisphere of the brain included in two image slices. The ten regions in the left hemisphere may be of the same types as the regions on the right hemisphere, and thus the ten regions on the left hemisphere may be considered to be complementary to respective ones of the regions on the right hemisphere, thereby forming complementary region pairs.

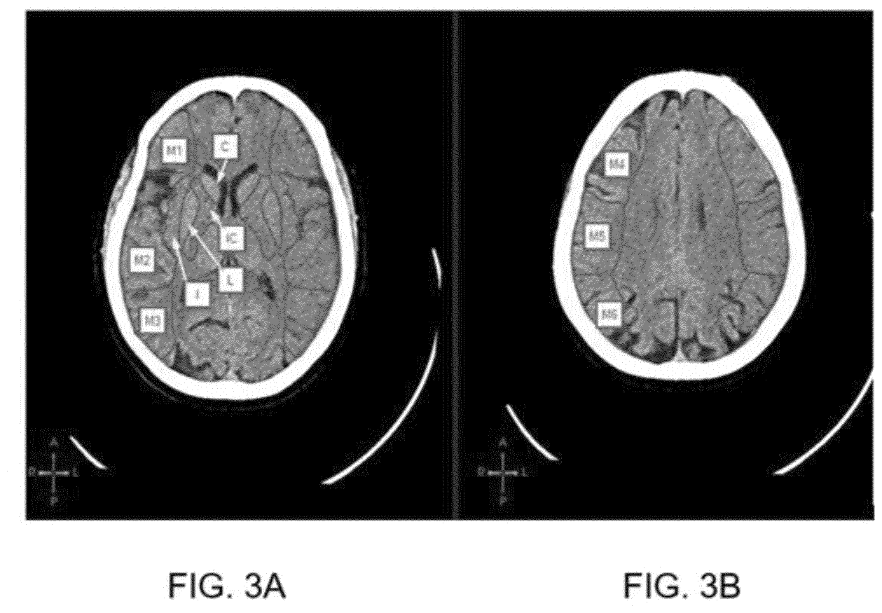

Table 1 identifies the ten regions in each hemisphere of the brain that correspond to the ASPECTS protocol. The ASPECTS protocol and its corresponding regions are discussed in https://linkinghub.elsevier.com/retrieve/pii/S0140673600022376, the contents of which are incorporated by reference herein for all purposes. Seven of the ten regions appear in the superior image slice (FIG. 3A) and the remaining three regions appear in the inferior image slice (FIG. 3B) shown on a per-hemisphere basis. Together, all ten regions may be considered candidate regions for lesions or other abnormalities that may adversely affect the brain.

TABLE 1

| Acronym | Region |
|---|---|
| C | Caudate head |
| IC | Internal Capsule |
| L | Lentiform Nucleus |
| I | Insular Ribbon |
| M1 | anterior MCA cortex, |
| | corresponding to frontal |
| | operculum |
| M2 | MCA cortex lateral to |
| | insular ribbon, |
| | corresponding to anterior |
| | temporal lobe |
| M3 | posterior MCA cortex, |
| | corresponding to posterior |
| | temporal lobe |
| M4 | anterior MCA territory, |
| | immediately superior to M1 |
| M5 | lateral MCA territory, |
| | immediately superior to M2 |
| M6 | posterior MCA territory, |
| | immediately superior to M3 |

In one embodiment, segmentation of the image slices may be performed automatically in 3D using a model-based approach. The model may process the images to identify (extract) and then generate overlay graphics outlining the contours of each of the ten regions of interest. Then, the model may label (e.g., see operation 225) each of the segmented regions as shown in FIGS. 3A and 3B. The contours may be matched with one another using, for example, multi-planar reformatting.

To train the model for the ASPECTS region segmentation, independent ground truth segmentations may be created using a plurality of datasets derived from the brains of different individuals. The datasets may include a combination of scans containing abnormalities (e.g., lesions from stroke victims) and scans of control patients without lesions. In-plane pixel spacing may vary among the images in the datasets. For example, in a practical application, the in-plan pixel spacing may vary in a range between 0.38 mm×0.38 mm and 0.58 mm×0.58 mm, whereas kVp may range between 100 and 140. The scans may have a slice thickness of a predetermined value (or within a predetermined range of values). One example of a slice thickness for the datasets is 3 mm.

Ground truth annotations for training the model-based region segmentation may be obtained by performing two operations, each harvesting previously available information for NCCT region annotation. Both operations may use, for example, a dedicated annotation application serving the following workflow. First the application may perform fully automatic adaptation of the initially trained model to the scan. Then, automatic multi-planar reformatting to the inferior ASPECTS slice may be performed. The multi-planar reformatting operation may be performed based on locating the centroid and principal components of the vertices in the mesh corresponding to the cortical areas M1-M3. The mesh may correspond, for example, to the pre-labeled version of the brain scan of FIG. 3C.

Next, interactive refinement and confirmation of the inferior ASPECTS slice may be performed, followed by removing (e.g., setting to invisible) region boundaries in different slices other than the inferior slice. The ASPECTS region boundaries in the inferior ASPECTS slice may then be corrected (e.g., interactively) and multi-planar reformatting to the superior ASPECTS slice may automatically be performed thereafter. This operation may be followed by interactive refinement and confirmation of the superior ASPECTS slice, after which region boundaries in different slices other than the inferior slice may be removed or otherwise set to invisible. Finally, correction of the ASPECTS region boundaries in the superior ASPECTS slice may be performed (e.g., interactively) in a similar manner.

The automatic segmentation of the image slices may be performed, for example, in accordance with the techniques described in WO 2020/109006, the contents of which are incorporated by reference herein.

At 225, a feature extraction operation is performed by the model to automatically label each of the contour regions in the superior and inferior image slices, for example, as indicated in Table 1. The feature extraction operation may be performed as follows. First, adaptation of the region boundaries in the image slices is performed to conform, for example, to a predetermined format. Positions of the adapted mesh vertices of the cortical areas M1-M3 (inferior slice) or M4-M6 (superior slice) are extracted. The cortical areas may be topologically defined, for example, as a 1 cm-thick stripe. A center of the viewing plane (e.g., focal point) may be computed as the centroid of the set of extracted vertices.

Next, an operation is performed to stack the extracted vertices into a matrix A and the three principal axes may be computed using, for example, a singular value decomposition (SVD) technique where A=USV′, in which U and V are orthogonal matrices and S is a diagonal matrix. The viewing plane normal may be extracted from the principal axes as the right-most vector of V. Then, a left-right (L-R) vector may be extracted as the mean between corresponding left and right vertices, since the bi-lateral areas are topologically symmetric by design. The A-P vector can then be finally computed based on a cross product of the right-most vector of V and the L-R vector using 3D algebra.

FIGS. 3A and 3B show examples of the superior image slice and the inferior image slice, respectively, that have been automatically annotated to include overlaid contours and labels for each of the regions. FIG. 3A shows the labels for the seven ASPECTS regions in the superior image slice, and FIG. 3B shows the labels for the remaining three ASPECTS regions in the inferior image slice. The inferior and superior image slices (with graphically added contours and labels) may be stored, as well as the corrected ASPECTS region as a mesh. (In one embodiment, the mesh may be considered to be graphically contoured and labelled image slices as shown, for example, in FIGS. 3A and 3B). As the number of training datasets increases, the model for performing automatic image segmentation may increase with accuracy.

At 230, intensity (HU) values are generated for the pixels in each of the labeled regions in the image slices. The intensity values may be, for example, Hounsfield Units (HU) values assigned, for example, in at least a predetermined HU range, e.g., an unsigned short value range. HU values provide an indication of the density of tissue expressed on a color scale or grayscale, e.g., various shades between black and white inclusive. Tissues with lower density may have darker shades (or intensity), while tissues with higher density may be expressed with lighter shades (or intensity). Thus, HU values effectively correspond to grayscale intensity values in a CT image that provide an indication of tissue density. In one embodiment, the HU values for each region of each image slice may be incorporated into a table and stored. For example, a first table may include HU values for the regions in the superior image slice on a per-hemisphere basis, and a second table may include HU values for the regions in the inferior image slice on a per-hemisphere basis.

At 235, intensity histograms are generated based on the HU values generated for the superior and inferior image slices. The intensity histograms may be generated on a bi-lateral basis, e.g., for each image slice one set of histograms may be generated for the regions in the left lateral portion of the brain and a complementary set of histograms may be generated for the regions in the right lateral portion of the brain. In this sense, the histograms generated for each image slice may be referred to as bi-lateral histograms which may be generated as follows.

In one embodiment, the intensity histograms are generated based on the HU values in the left and right lateral portions of the superior image slice. The set of histograms generated for the left lateral portion include seven histograms, one histogram for each of the seven APSECTS regions labeled in the superior image slice. The histogram generated for a first region of the seven regions in the left lateral portion of the brain provides an indication of HU values of the pixels in first region. For example, a first number of pixels in the first region may have a first HU value, a second number of pixels in the first region may have a second HU value, and so on. These numbers form a distribution of HU values in the histogram that may be used as a basis for determining the character of the tissue and (if included) lesions or other abnormalities in that region. Histograms for the remaining six regions in the left lateral portion of superior image slice may then be generated in like manner.

Once all of the histograms for the regions in the left lateral portion are generated, histograms are generated in like manner for each of the seven regions in the right lateral portion of the superior image slice. Thus, operation 235 produces a total of 14 histograms, seven histograms for respective regions in the left lateral portion and seven complementary histograms for respective regions in the right lateral portion of the superior image slice.

The range of HU values may be the full range of HU values or a predetermined subset of values within the full range which, for example, may be considered relevant to the particular type(s) of brain abnormality of interest. The subset of values may, for example, correspond to a predetermined number of bins into which the full range of HU values are partitioned. For example, in one implementation the intensity histograms may have values limited to a range of between an HU value of 10 and an HU value of 60 spread across 25 bins with each bin have a size of 2 HUs. This range may be considered suitable for some applications, e.g., the lower HU boundary value of 10 excludes large parts of the CSF, where the upper HU boundary value of 60 completely includes gray matter but excludes calcifications and hemorrhagic lesions. The histogram data may be set based on a different range of HU values in another embodiment.

The set of histograms generated for the right lateral portion include three histograms, one histogram for each of the three APSECTS regions labeled in the inferior image slice. The histogram generated for a first region of the three regions in the left lateral portion of the brain provides an indication of HU values of the pixels in that first region. These numbers form a distribution of HU values in the histogram that may be used as a basis for determining the character of the tissue and (if included) lesions or other abnormalities in that region. Histograms for the remaining two regions in the left lateral portion of inferior image slice may be generated in like manner.

Once all of the histograms for the regions in the left lateral portion are generated, histograms are generated in like manner for each of the three regions in the right lateral portion of the inferior image slice. Thus, operation 340 produces a total of 6 histograms, three histograms for respective regions in the left lateral portion and three complementary histograms for respective regions in the right lateral portion of the inferior image slice. The same range used to generate the histograms for the superior image slice may be used for generating the histograms in the inferior image slice.

Based on operations 235, a total of twenty histograms may be generated for the superior and inferior image slices that provide a set of comprehensive histogram data to be used to identify a lesion. Because the ten histograms for the left brain portion are complementary to the ten histograms for the right brain portion across the two image slices, the histograms for each complementary pair will have different distributions of HU values when one of the regions has a lesion and the other does not.

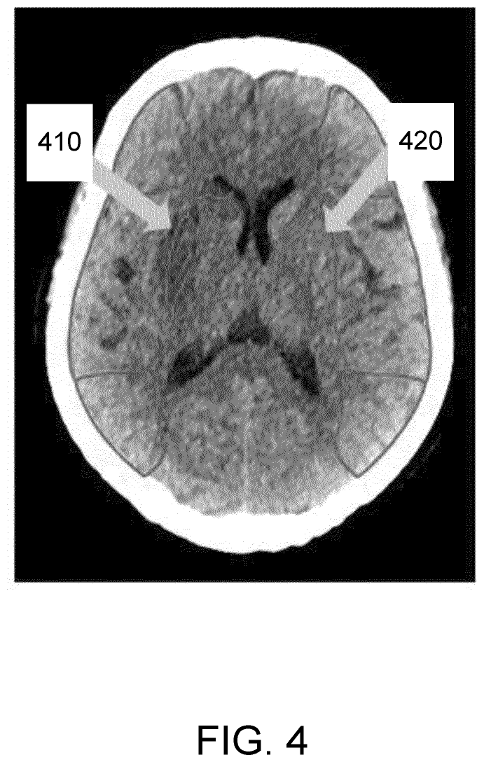

FIG. 4 shows, for example, an example of a superior image slice of a patient having an ischemic lesion in the Lentiform Nucleus (L) region 410 of the left lateral portion of the brain. The L region in the right lateral portion 420 of the brain (which is complementary to region 410) does not have a lesion. A lesion includes a different type of tissue with a different density from brain tissue, and therefore the lesion tissue will exhibit a different scan intensity than normal brain tissue. As a result, the histogram distribution of HU values in region 410 will be substantially different from the histogram distribution of HU values in region 420. This difference may serve as a basis for generating derivative histogram data that may identify the lesion.

At 240, derivative histogram data is generated based on the histograms generated in operation 235. The derivative histogram data may include, for example, a plurality of difference histograms for respective ones of the ten ASPECTS regions labeled in the superior and inferior image slices. Referring to 240A, in one embodiment each difference histogram may be generated based on a difference between the histogram of HU values of one region in the left lateral portion of the brain and the histogram of HU values of the complementary region in the right lateral portion of the brain, e.g., the histogram for the L region in the left lateral portion is subtracted from the histogram for the L region in the right lateral portion. Thus, ten difference histograms are generated for respective ones of the ten regions labeled in the superior and inferior image slices.

The difference histograms provide a substantive (quantitative and qualitative) indication of how different the intensity values are at each complementary pair of locations in the brain scans. For example, when a difference histogram has difference values that fall below a predetermined threshold or are in a first range (or otherwise demonstrate a first type of pattern), the difference histogram may be used to infer that the complementary regions in the relevant image slice do not have an abnormality. When a difference histogram has difference values that are above the predetermined threshold or are in a second range (or otherwise demonstrate a second type of pattern), the difference histogram may be used to infer that at least one of the complementary regions in the relevant image slice is a candidate for containing abnormality, e.g., a lesion.

Referring to 240B, the first histogram for the left lateral region may be compared to a first reference histogram and the second histogram for the complementary right lateral region may be compared to a second reference histogram for that region. The first reference histogram may have intensity values indicative of all normal brain tissue (e.g., health tissue or at least tissue without a lesion or a particular type of lesion) in the corresponding brain region. The second reference histogram may have intensity values indicative of normal healthy brain tissue (e.g., health tissue or at least tissue without a lesion or a particular type of lesion) in the corresponding complementary region, e.g., the same region on the opposing hemisphere.

Thus, in 240B, a first difference histogram is generated based on a comparison of the first histogram and the first reference histogram (e.g., subtracting intensity values in the first histogram from intensity values in the first reference histogram), and a second difference histogram may be generated based on a comparison of the second histogram and the second reference histogram (e.g., subtracting intensity values in the second histogram from intensity values in the second reference histogram).

In another embodiment, the method may extend the regions of interest from 2D (ASPECT areas) to 3D (volumes containing voxels in different (e.g., adjacent) image slices that also belong to the ASPECT region. This embodiment ensures that the histograms caused by lesions that are more apparent in the vicinity (below or above) the 2D ASPECT area. The system and method embodiments may then be applied in an analogous manner to these 3D image slices.

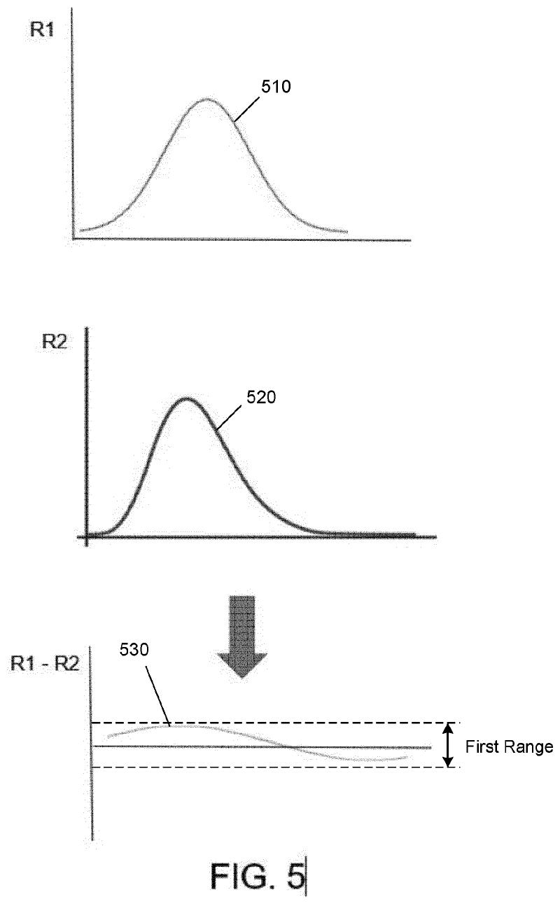

FIG. 5 shows an example of a difference histogram (generalized to a curve) 530 generated based on a histogram 510 (generalized to a curve) corresponding to a region in the left lateral portion of an image slice and a histogram 520 (generalized to a curve) corresponding to the same (or complementary) region in the right lateral portion of the image slice. In the example shown in FIG. 5, both complementary regions do not have a lesion. Without a lesion (e.g., with healthy brain tissue) in this region pair, both histograms 510 and 520 may have similar values and peaks in substantially the same range(s) of HU values. As a result, the difference histogram 530 may have values that fall below a threshold or which lie in a first range. For purposes of illustration, histogram 510 is labeled R1 and histogram 520 is labeled R2. Thus, difference histogram 530 may be computed by the equation: R1-R2, thus accounting for the negative difference values in histogram 530.

Referring again to FIG. 4, an example of a superior image slice is shown which includes an ischemic lesion in the left lateral portion of the brain. In this example, the lesion (shown by arrow 410) is located in the lentiform nucleus (L) region. The lentiform nucleus region in the complementary lateral portion of the brain (shown by arrow 420) is normal in that it does not include a lesion. As is evident from a comparison of the regions in this image slice, the L region in the left lateral portion of the image has an intensity which is different from (e.g., darker than) the intensity of the L region in the right lateral portion of the image. The difference in these intensities produce different HU values for these L regions which will be reflected in the histograms generated for these different L areas.

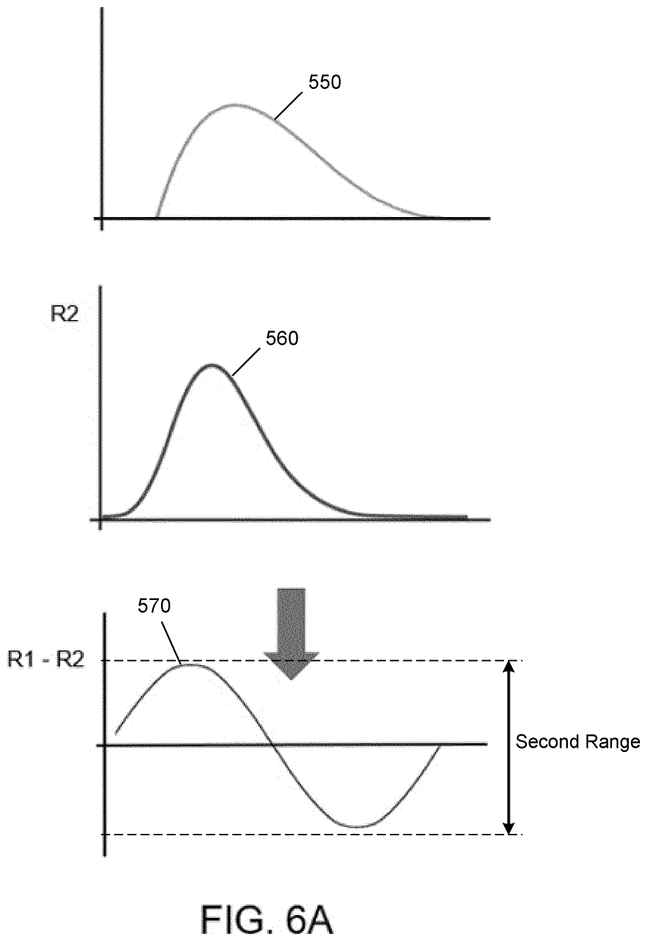

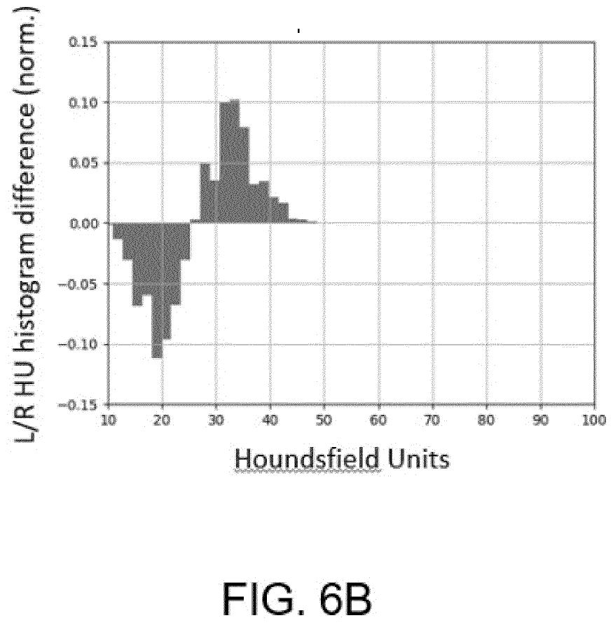

FIG. 6A shows an example of a difference histogram (generalized to a curve) 570 which may be generated based on the lesion shown in the image slice of FIG. 4. As shown in FIG. 6A, the difference histogram 550 is generated based on a histogram (generalized to a curve) 550 corresponding to the L region in the left lateral portion of the superior image slice and a histogram (generalized to a curve) 560 corresponding to the complementary L region in the right lateral portion of the same image slice. As indicated, the L region corresponding to histogram 550 has a lesion and L region 560 does not. As a result, difference histogram 570 will have values that exceed the threshold or which otherwise fall within a second range, which, for example, may be greater than the first range. An example the difference histogram, not generalized by a curve, is shown in FIG. 6B. In one embodiment, the shape of a curve and/or the extent of the disparity of values in the difference histogram may form a pattern that is indicative of whether or not a lesion exists in one or more of the regions of the image slices.

In addition, the method may include an optional extraction operation which involves determining regions which have a certain probability of having a lesion and ones that do not. This determination may be made based on the difference histograms. Regions that are deemed to have a higher probability of having a lesion may be considered as candidate regions for further evaluation.

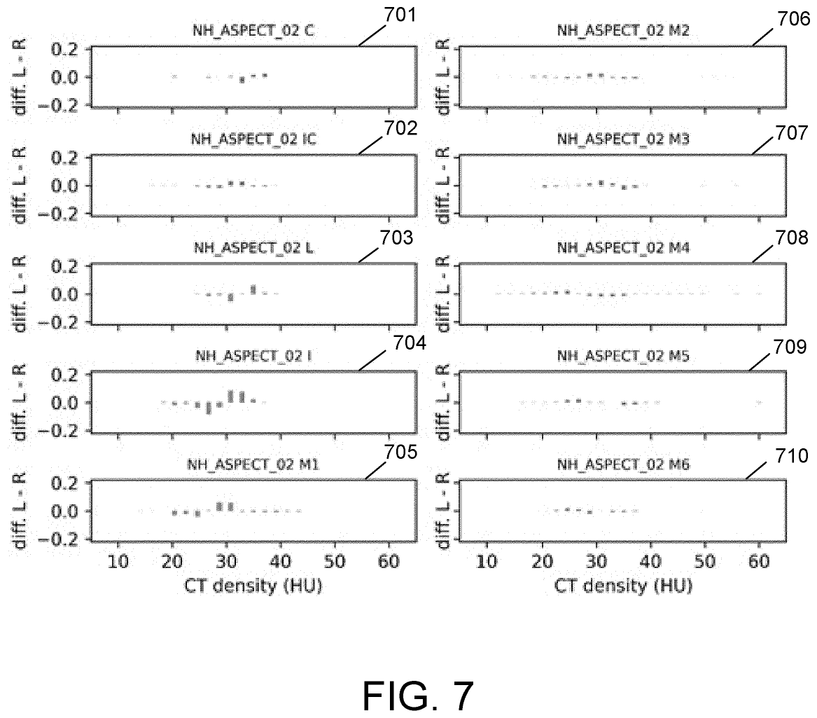

FIG. 7 shows examples of difference histograms 701 to 710 generated for all ten ASPECTS regions. In this example, difference histograms 703 to 706 have values (or patterns) that may qualify as candidate regions for possibly containing lesions. The remaining difference histograms may correspond to non-lesion representations in corresponding ones of the regions.

The generation of difference histograms for each of the regions in the image slices, therefore, effectively serves as a classifier that may be used to distinguish between candidate regions that may include a lesion requiring further evaluation. In one embodiment discussed in greater detail below, feature vectors may be generated based on the difference histograms for input into a machine-learning model (e.g., neural network) which may operate as a classifier to confirm candidate lesions determined based on the data in the difference histograms. In one embodiment, prior to generation of the difference histograms, each histogram may be normalized with respect to the number of voxels to account for differences in the sizes of regions whose histograms are to be compared.

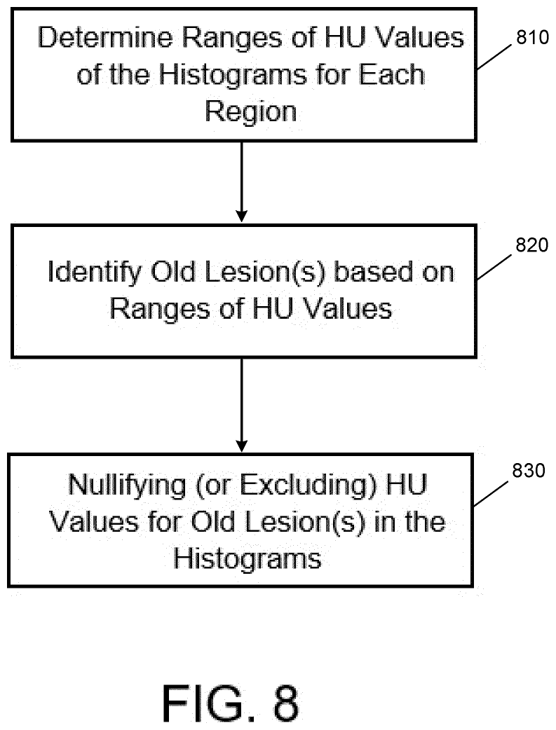

FIG. 8 shows another embodiment of a method for automatically identifying abnormalities in the brain scans of patients. This method is like the method of FIG. 2 but may be supplemented with a filtering operation that involves distinguishing a new lesion from an old lesion in one or more of the superior or inferior image slices. This may be accomplished, for example, by identifying the existence of two or more lesions in a given ASPECTS region, or in different ASPECTS regions, identifying at least one new lesion and at least one old lesion, and the discarding data (e.g., HU values) in the resulting histogram(s) corresponding to the old lesion and focusing only on the new lesion, for purposes of generating difference histograms and for applying a model to determine whether a new lesion exists.

Ischemic stroke lesions may be classified into three categories: 1) Acute corresponding to a stroke that occurred in a patient from 0 to 24 hours, 2) Subacute corresponding to a stroke that is older than 24 hours, and 3) Old corresponding to a lesion that occurred from a previous stroke, which, for example, may be months or years old. In practice, the type (or timespan) of a lesion cannot alone be determined from visual inspection of a non-contrast CT because additional clinical information may be required. Because time is of the essence in a stroke victim, this additional clinical information may not be readily available, which could introduce delays in treatment.

For purposes of providing optimal treatment, the present method embodiment may automatically evaluate and determine, in just seconds, whether a lesion is likely a new lesion (acute or subacute) from an old lesion. This is especially beneficial when multiple lesions appear in the image slices, either within the same or different ASPECTS regions.

In one embodiment, the method of FIG. 8 may therefore include a number of additional operations in the method of FIG. 2. These operations may be performed, for example, during or after operation 235 is performed. As previously discussed, during operation 235 intensity histograms are generated based on the HU values in the left and right lateral portions of the superior image slice, and intensity histograms are generated based on the HU values in the left and right lateral portions of the inferior image slice.

Old lesions will generate HU values with a concentration in a certain range, while new lesions will generate HU values with a concentration in a different range. The difference in these intensity values may be attributed to, for example, calcification and other effects due to aging.

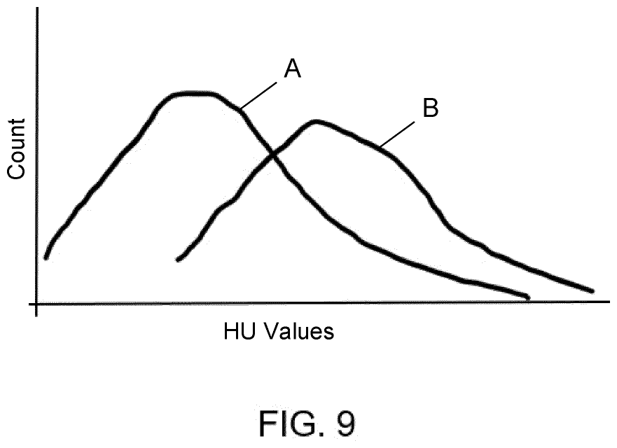

FIG. 9 shows an example of the difference in the histogram curve A generated by an old lesion and the histogram curve B generated by a new lesion. As shown, histogram curve A has a concentration of lower HU values than does histogram curve B. The concentration of lower HU values for curve A may be ones exhibited by an old lesion and thus may be used as a basis for identifying curve A as potentially corresponding to an old lesion. In contrast, histogram curve B is shifted to the right relative to curve A and thus has a concentration of higher HU values, e.g., ones that may be exhibited by a new lesion. These higher HU values may therefore be used as a basis for identifying curve B as potentially corresponding to a new lesion.

Referring to FIG. 8, the method may therefore include evaluating the ranges of HU values of the histograms generated for each of the regions (810), identifying a potential lesion as an old lesion based on determining a concentration of low HU values (820), and then deleting or otherwise nullifying or excluding the histogram values that correspond to the potentially old lesion (830). With these values deleted or nullified, the method of FIG. 2 may continue with generating difference histograms for the corresponding region(s). One or more of the operations of FIG. 8 may be performed by the discrimination logic 40 of FIG. 1.

### Classification Model

A classification model may be used to classify the difference histograms generated in the aforementioned operations. The classification model may be implemented in various forms. In one embodiment, the classification model may be a binary classifier which outputs a decision indicating (with at least a certain probability) whether a candidate region has a lesion or does not have a lesion. A model based on use of a convolutional neural network (CNN) is discussed as one non-limiting example of evaluating the difference histograms in order to identify and classify whether or not the candidate regions likely have a lesion.

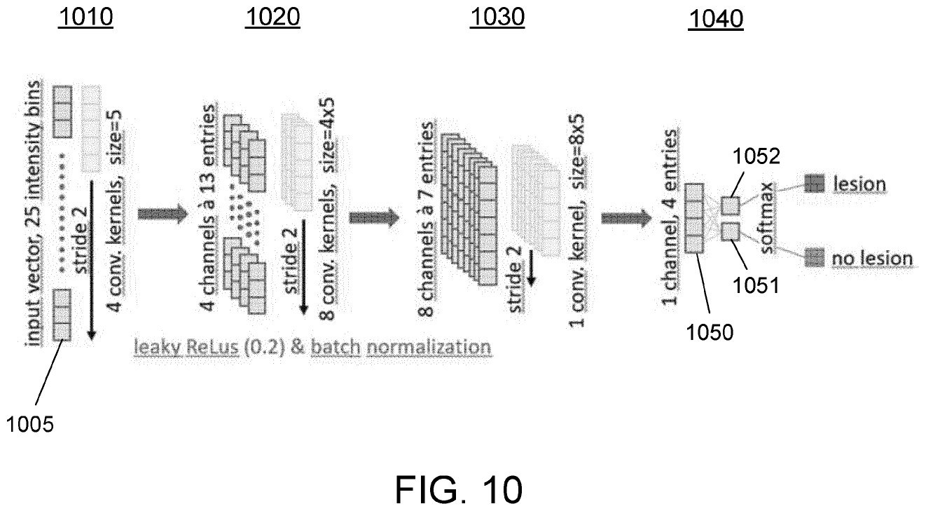

FIG. 10 shows an embodiment of the CNN model used to generate the binary lesion decision. The CNN model includes a first layer 1010, which may be a convolutional layer that receives input vectors generated, for example, based on the difference histograms produced for each of the ten ASPECTS regions in the image slices. The first convolutional layer may have a first number of kernels and generates a multi-dimensional vector of a first size. This vector is input into a second layer 1020, which is another convolutional layer with a second number of kernels that generates another multi-dimensional vector of a second size different from the first size. The vector output from the second convolutional layer may be input into a third layer 1030, which is another convolutional layer with a third number of kernels that generates another vector of a third size. The vector output from the third convolutional layer is input into a third layer 1040, which is a fully connected layer with a predetermined number of input nodes and output nodes respectively representing the class probabilities (lesion/no lesion) corresponding to the decision for the region corresponding to the initial input feature vector. As will be described below, in one embodiment the output vectors of the convolutional layers may be passed through an activation function prior to being input into a subsequent layer.

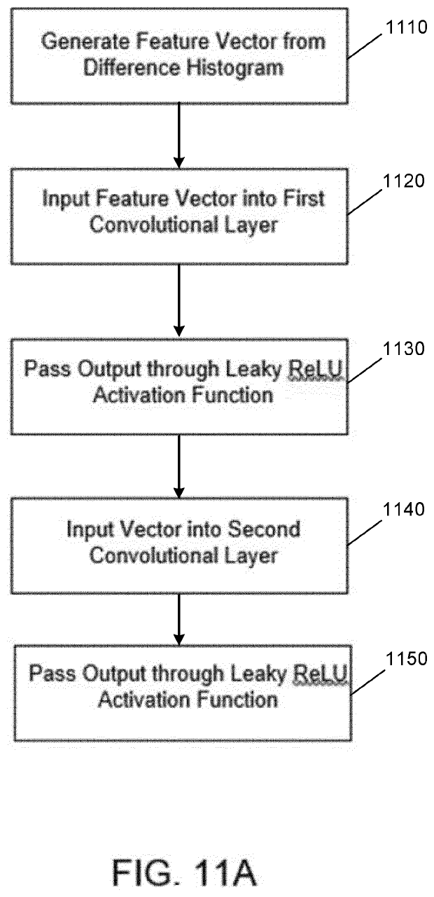

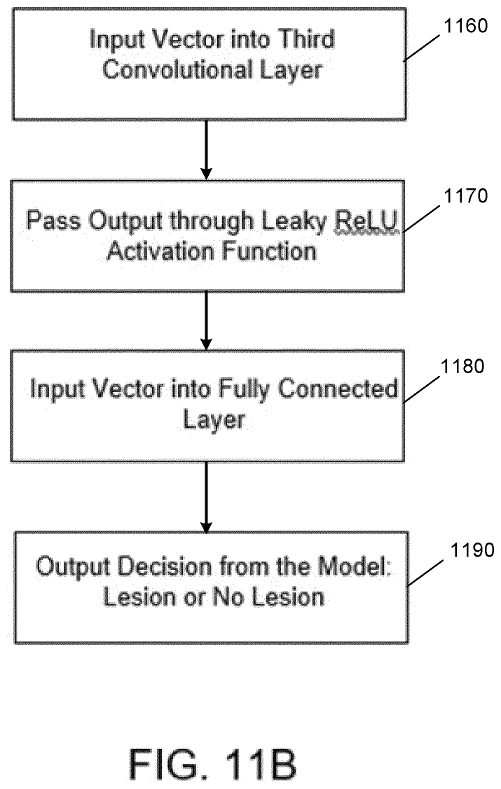

FIGS. 11A and 11B show operations included in one embodiment of a method which uses a CNN model to generate a binary lesion decision. The method may use the CNN model of FIG. 10 or a different CNN model in another embodiment. For illustrative purposes, the method will be described as using the CNN model of FIG. 10 having a particular configuration. This configuration and/or its example values may be changed in other embodiments depending, for example, on the datasets used to train the model. Also, in other embodiments a different number of convolutional layers may be used and/or the layers may be configured in a different manner, for example, to satisfy requirements of the intended application.

Referring to FIG. 11A, the method includes, at 1110, generating a feature vector 1005 for each difference histogram generated as candidates to have a lesion. The feature vector may be, for example, a one-dimensional vector with a predetermined number of entries (e.g., 25 entries) corresponding to the number of intensity bins. For example, when the difference histogram is based on a range of 50 HU values as previously explained, 25 intensity bins (or entries) may be generated with each bin (or stride) spanning 2 HU values each. In another embodiment, the feature vector may be generated based on a difference histogram produced from another operation (one different from operation 450) and/or a different number of entries or kernels may be used.

At 1120, the feature vector 1005 is input into the first convolutional layer 1010, which is configured to have a first number (e.g., 4) convolutional kernels with a kernel size of a first size (e.g., 5) each. The convolutional kernels effectively serve as sliding windows or filters, each of which may include, for example, a matrix of values (in this example, 5 values per window) that are multiplied by the values in the feature vector. For example, the first convolutional layer 1010 may use a first window to perform a convolutional operation on the values of the feature vector 1005. The windows corresponding to the three remaining kernels (having at least one value different from the first kernel) may be used to perform additional convolutional operations on the values of the feature vector 1005. When the stride is set to 2, the output of the first convolutional layer is a four-dimensional vector of size 4×13 (zero-padding with size 2).

In one embodiment, the internal values (e.g., weights) of the convolutional layers may be determined via AI training. The actual setup (e.g., network topology) in which the number of kernels and their sizes is shown is based on the performance of internal experiments. In some embodiments, variants of the neural network may be used to generate the model output.

By multiplying the feature vector values by corresponding values in the kernels, kernels are used to effectively test the feature vector to provide an indication of the information embedded in the underlying difference histogram. The outputs of the convolutional operations will be different because of the different values in the kernels. Also, different feature vectors (based on different difference histograms) will generate different outputs from the first convolutional layer for the same kernels. In some cases, the output of the first convolutional layer may provide an indication of whether or not a lesion exists in the region corresponding to the feature vector. However, one or more additional convolutional layers (e.g., at least a second) may be included to provide a more accurate decision.

At 1130, the four-dimensional vector output of the first convolutional layer may be passed through a leaky rectified linear unit (leaky ReLU) activation function to reduce the size or otherwise process the vector to have a predetermined form. The leaky ReLU activation function may be implemented using a predetermined slope, which, for example, may be 0.2 for some applications. The leaky ReLU activation function may be expressed as f(x)=1 (x<0) (αx)+1 (x>=0) (x), where α is a predetermined constant. In one embodiment, operation 1130 may be considered to be optional.

At 1140, the feature vector output from the first convolutional layer (as optionally passed through the leaky ReLU activation function) is input into the second convolutional layer. This convolutional layer may have a second number of kernels with the first kernel size. In one embodiment, the second number of kernels may be 8 kernels and the kernel size may be 5 (×4). The same stride of 2 as used in the first convolutional layer may be used in the second convolutional layer.

At 1150, the vector output from the second convolutional layer may optionally be passed through a leaky activiation function to reduce the output size of the vector. For example, the vector may be reduced to a size of 8×7.

Referring to FIG. 11B, the method includes, at 1160, the feature vector output from the second convolutional layer (as optionally passed through the leaky ReLU activation function) is input into the third convolutional layer. In one embodiment, the third convolutional layer may have one kernel with the same kernel size 5 (×8).

At 1170, the vector output from the third convolutional layer may optionally be passed through another leaky ReLU activation function to reduce the output side of the vector to 4 (×8).

At 1180, the vector output from the third convolutional layer (as optionally passed through the leaky ReLU activation function) is input into the fourth layer, which may be a fully connected layer with 4 input nodes 1050 and 2 output nodes 1051 and 1052 representing class probabilities (lesion/no lesion) corresponding to the decision. The class probabilities may be normalized, for example, using a softmax function such that the sum of the probabilities equals one.

At 1190, the output nodes 1051 and 1052 output their respective probabilities the decision of no lesion and lesion. The output node having the larger probability may serve as the decision output from the model. In this manner, the CNN model thus operates as a classifier generating data from the output nodes that may be used as a basis to indicate a decision of a lesion or no lesion. For example, when the probability threshold that a lesion exists (generated from output node 1052) equals or exceeds a predetermined percentage (e.g., 50%), the decision generated from the model may indicate a lesion. Otherwise, the decision may indicate no lesion (because the probability of output node 1051 (no lesion)<the probability of output node 1052 (lesion)).

When the difference histograms are generated based on reference histograms as previously discussed, a difference histogram is generated for each region relative to the reference histograms. In this case, the model may be trained with datasets generated relative to the reference histograms for each ASPECTS region, e.g., one reference histogram for the region on the left lateral portion of the image slice and another reference histogram for the complementary region on the right lateral portion of the image slice. The reference histograms may represent no-lesion data for their respective regions.

Irrespective of the way the difference histograms are generated, feature vectors corresponding to the difference histograms may be generated and input into the model to provide an indication of whether each corresponding ASPECT region likely has a lesion. In one embodiment, the difference histograms for all ten regions may be input through the model. This would alleviate the need to perform a preliminary filtering operation to discard non-candidate regions (e.g., ones having different histograms within only the first range as previously discussed) and thus would result in generating probability decisions for all ten regions. In such a case, a comprehensive set of data may be provided for healthcare professionals for review and use in determining treatment options.

Training of the model is performed, at least in part, based on the manner in which the difference histograms are generated. In one embodiment, the neural network may have a total of 269 weights (e.g., trainable parameters) to ensure that overfitting will not be an issue. Training of the neural network may be performed based on, for example, PyTorch as a backend, by using its built-in cross entropy loss function and by using loss weights of 1 and 3 for the “no lesion” and “lesion” class to account for class imbalance in the training data set. During training, an Adam optimizer may be used with, for example, a learning rate of 2×10⁴ for 300 epochs. Also, batch normalization on the second and third convolutional layers may be employed and a batch size of 16 (histograms) may be used.

During training, some data augmentation may be performed by adding random noise to the difference histogram (e.g., sampled from a normal distribution centered at zero and with an amplitude of 3×10⁵) and by reversing the sign of the difference histogram (that would correspond to a flip of left and right on the image) with a probability of 50%.

The data used for training the CNN lesion detection classifier may include over 100 non-contrast CT datasets, e.g., 115 datasets were used in an actual case. Some of these datasets may correspond to datasets used to train the model for performing region segmentation. kVp values may, for example, be distributed from 100 (n=25) over 120 (n=45) to 140 (n=45).

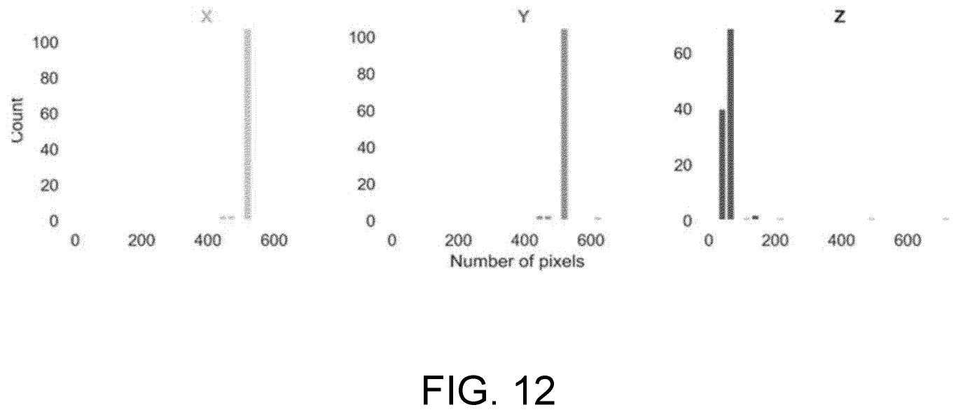

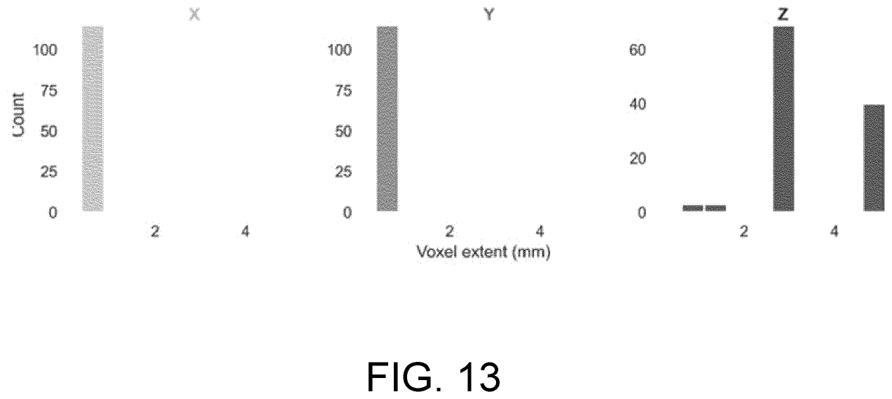

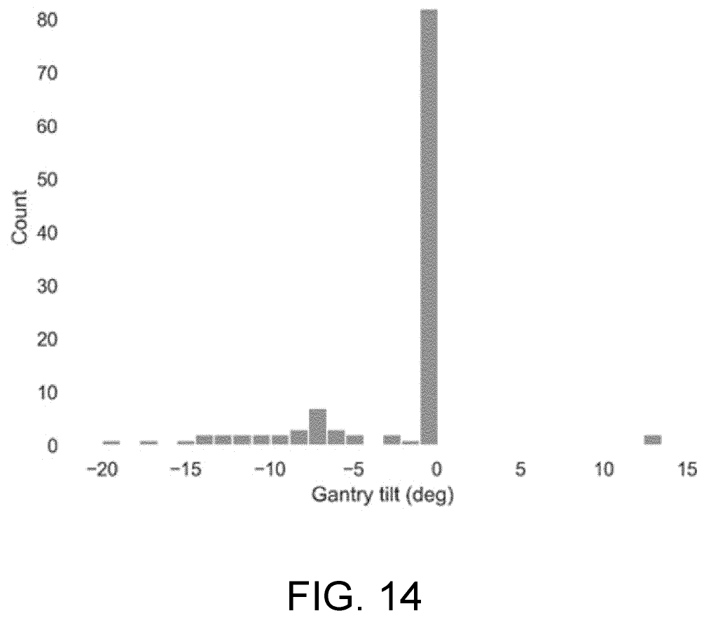

FIGS. 12, 13, and 14 show examples of the variability in image parameters that may be used during training. In FIG. 12, image dimensions range between (mean±standard deviation) 508.71±13.79 in the x direction, 511.26±21.88 in the y-direction, and 64.27±76.18 in the z-direction. In FIG. 13, Voxel spacings are (mean±standard deviation) 0.46±0.04 mm in the x-direction, 0.46±0.04 mm in the y-direction and 3.59±1.13 mm in the z-direction. In FIG. 14, in the CNN classification training set 33 cases (actual example) or more may be used with or without a gantry tilt.

While some of the system and method embodiments have been described as identifying lesions and other brain abnormalities using two images (e.g., an inferior image slice and a superior image slice), other embodiments may be implemented to identify such abnormalities using only one image slice. In this case, all ten of the APSECTS regions may not be taken into consideration. However, the embodiments may still be implemented in a meaning way as previously described relative to one image slice to identify ischemic lesions and other brain abnormalities.

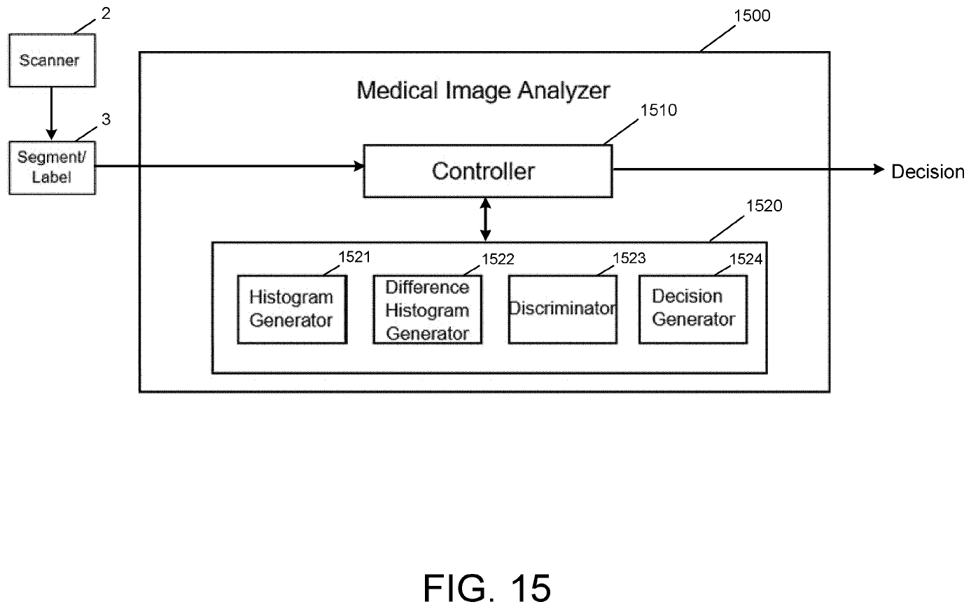

FIG. 15 shows an embodiment of a medical image analyzer 1500 which may implement the method embodiments described herein. Referring to FIG. 15, medical image analyzer 1500 includes a controller 1510 and a memory 1520. The controller may execute instructions stored in the memory for performing the operations and method described herein. In this embodiment, the instructions stored in memory 1520 may include a first set of instructions 1521 that implement a histogram generator, a second set of instructions 1522 that implement a difference histogram generator, a third set of instructions that implement a discriminator 1523, and a fourth set of instructions 1524 that implement a decision generator. These sets of instructions may respectively perform, for example, the operations of the features in the medical image analyzer of FIG. 1.

In another embodiment, two histograms may be directly fed into a classifier instead of the difference histogram. In this situation, the difference histogram is implicitly calculated by the trained model, that is trained using pairs of histograms.

In accordance with one or more of the aforementioned embodiments, the methods, processes, and/or operations described herein may be performed by code or instructions to be executed by a computer, processor, controller, or other signal processing device. The computer, processor, controller, or other signal processing device may be those described herein or one in addition to the elements described herein. Because the algorithms that form the basis of the methods (or operations of the computer, processor, controller, or other signal processing device) are described in detail, the code or instructions for implementing the operations of the method embodiments may transform the computer, processor, controller, or other signal processing device into a special-purpose processor for performing the methods described herein.

Also, another embodiment may include a computer-readable medium, e.g., a non-transitory computer-readable medium, for storing the code or instructions described above. The computer-readable medium may be a volatile or non-volatile memory or other storage device, which may be removably or fixedly coupled to the computer, processor, controller, or other signal processing device which is to execute the code or instructions for performing the operations of the system and method embodiments described herein.

The processors, systems, controllers, and other signal-generating and signal-processing features of the embodiments described herein may be implemented in logic which, for example, may include hardware, software, or both. When implemented at least partially in hardware, the processors, systems, controllers, and other signal-generating and signal-processing features may be, for example, any one of a variety of integrated circuits including but not limited to an application-specific integrated circuit, a field-programmable gate array, a combination of logic gates, a system-on-chip, a microprocessor, or another type of processing or control circuit.

When implemented in at least partially in software, the processors, systems, controllers, and other signal-generating and signal-processing features may include, for example, a memory or other storage device for storing code or instructions to be executed, for example, by a computer, processor, microprocessor, controller, or other signal processing device. The computer, processor, microprocessor, controller, or other signal processing device may be those described herein or one in addition to the elements described herein. Because the algorithms that form the basis of the methods (or operations of the computer, processor, microprocessor, controller, or other signal processing device) are described in detail, the code or instructions for implementing the operations of the method embodiments may transform the computer, processor, controller, or other signal processing device into a special-purpose processor for performing the methods described herein.

The benefits, advantages, solutions to problems, and any element(s) that may cause any benefit, advantage, or solution to occur or become more pronounced are not to be construed as a critical, required, or essential features or elements of any or all the claims. The invention is defined solely by the appended claims including any amendments made during the pendency of this application and all equivalents of those claims as issued.

Although the various exemplary embodiments have been described in detail with particular reference to certain exemplary aspects thereof, it should be understood that the invention is capable of other example embodiments and its details are capable of modifications in various obvious respects. As is apparent to those skilled in the art, variations and modifications can be affected while remaining within the spirit and scope of the invention. The embodiments may be combined to form additional embodiments. Accordingly, the foregoing disclosure, description, and figures are for illustrative purposes only and do not in any way limit the invention, which is defined by the claims.

1. A method for processing medical information, comprising:
receiving an image slice of a brain including segmented regions;
forming a first histogram of intensity values for the image slice;
forming a second histogram of intensity values for the image slice; and
determining an abnormality in the image slice based on the first histogram and the second histogram, wherein the first histogram corresponds to a first segmented region in a first portion of the image slice and the second histogram corresponds to a second segmented region in a second portion of the image slice which is complementary to the first portion.

2. The method of claim 1, further comprising:
generating at least one difference histogram based on the first histogram and the second histogram, wherein determining the abnormality in the image slice is based on the at least one difference histogram.

3. The method of claim 2, wherein determining the abnormality includes:
identifying that the at least one difference histogram has values in a range; and
determining the abnormality based on the values of the at least one difference histogram in the range.

4. The method of claim 2, wherein determining the abnormality includes:
identifying that the at least one difference histogram has one or more values that exceed a predetermined reference value; and
determining the abnormality based on the one or more values of the at least one difference histogram exceeding the predetermined reference value.

5. The method of claim 2, wherein generating the at least one difference histogram includes subtracting the intensity values of the first histogram from the intensity values of the second histogram.

6. The method of claim 2, wherein generating the at least one difference histogram includes:
generating a first difference histogram based on a difference between the first histogram and a first reference histogram; and
generating a second difference histogram based on a difference between the second histogram and a second reference histogram.

7. The method of claim 6, wherein:
the first reference histogram is indicative of brain tissue without a lesion in the first segmented region, and
the second reference histogram is indicative of brain tissue without a lesion in the second segmented region.

8. The method of claim 2, wherein determining the abnormality includes:
generating a feature vector based on the at least one difference histogram;
inputting the feature vector into a classifier model; and
predicting the abnormality based on an output of the classifier model.

9. The method of claim 2, further comprising:
determining whether the first histogram has a concentration of intensity values in a predetermined range; and
identifying that the image slice has an old abnormality when the first histogram has the concentration of intensity values in the predetermined range.

10. The method of claim 9, further comprising:
generating the difference histogram based on an exclusion of the intensity values in the predetermined range of the first histogram corresponding to the old abnormality.

11. The method of claim 1, wherein the first segmented region and the second segmented region are complementary ASPECTS regions.

12. A system for processing medical information, comprising:
a histogram generator configured to generate a first histogram of intensity values for an image slice and a second histogram of intensity values for the image slice, the image slice including segmented regions of a brain;
and
a decision engine configured to determine an abnormality in the image slice based on the first histogram and the second histogram, wherein the first histogram corresponds to a first segmented region in a first portion of the image slice and the second histogram corresponds to a second segmented region in a second portion of the image slice which is complementary to the first portion.

13. The system of claim 12, further comprising:
difference logic configured to generate at least one difference histogram based on the first histogram and the second histogram, wherein the decision engine is configured to determine an abnormality in the image slice based on the at least one difference histogram,

14. The system of claim 13, wherein the decision engine is configured to determine the abnormality by:
identifying that the at least one difference histogram has values in a range; and
determining the abnormality based on the values of the at least one difference histogram in the range.

15. The system of any of claim 13, wherein the decision engine is configured to determine the abnormality by:
identifying that the at least one difference histogram has one or more values that exceed a predetermined reference value; and
determining the abnormality based on the one or more values of the at least one difference histogram exceeding the predetermined reference value.

16. The system of claim 13, wherein the difference logic is configured to generate the at least one difference histogram by subtracting the intensity values of the first histogram from the intensity values of the second histogram.

17. The system of claim 13, wherein the difference logic is configured to:
generate a first difference histogram based on a difference between the first histogram and a first reference histogram; and
generate a second difference histogram based on a difference between the second histogram and a second reference histogram.

18. (canceled)

19. The system of claim 13, wherein the decision engine is configured to determine the abnormality by:
generating a feature vector based on the at least one difference histogram;
inputting the feature vector into a classifier model; and
predicting the abnormality based on an output of the classifier model.

20. The system of claim 13, wherein the difference logic is configured to generate the at least one difference histogram by subtracting the intensity values of the first histogram from the intensity values of the second histogram.

21. The system of claim 12, further comprising:
a discrimination logic configured to:
determine whether the first histogram has a concentration of intensity values in a predetermined range; and
identify that the image slice has an old abnormality when the first histogram has the concentration of intensity values in the predetermined range.

22. The system of claim 21, wherein the difference logic is configured to generate the at least one difference histogram based on an exclusion of the intensity values in the predetermined range of the first histogram corresponding to the old abnormality.

23. A computer program comprising instructions which, when the program is executed by a computer, cause the computer to carry out the steps of claim 1.

24. (canceled)
